(19) United States
(12) Patent Application Publication
(10) Pub. No.: US 2006/0052357 A1
(43) Pub. Date: Mar. 9, 2006

(54) Organometallic anti-tumour agent

(75) Inventor: Valerij Tatarsky, Potsdam (DE)

Correspondence Address: NORRIS, MCLAUGHLIN & MARCUS, P.A., 875 THIRD AVE, 18TH FLOOR, NEW YORK, NY, 10022, US

(73) Assignee: HAEMATO-BASICS GMBH, LUCKENWALDE (DE)

(21) Appl. No.: 10/519,419

(22) PCT Filed: Jul. 2, 2002
(86) PCT No.: PCT/EP02/07299

§ 371 Date: Dec. 22, 2004

(30) Foreign Application Priority Data
Jul. 3, 2001 (UZ) IDP 2001 0552
Jan. 17, 2002 (DE) 102 01 693.3

(51) Int. Cl. C07F 15/02; C07F 15/04; C07F 15/06; A61K 31/555

(52) U.S. Cl. 514/184; 544/225; 546/2; 549/205

(57) ABSTRACT

The present invention relates to complexes of the general formula
D₂—M—T where D is a β-diketone, M is a metal atom and T is a substance having at least one N-, O- or S-containing group, and to the use thereof as antitumor agents.

The present invention relates to complexes of the general formula
D₂—M—T
where

D is a β-diketone, M is a metal atom and T is a substance having at least one N-, O- or S-containing group, and to the use thereof as antitumor agents.

Each year 300 000 people in Germany develop cancer, and about one in five Germans dies from a tumorous disease, a number which will undoubtedly increase in future years. About 55% of all cancer patients are diagnosed with a tumorous disease which is still localized, whereas a tumorous disease which is already advanced and metastasizing is present in the remaining 45%. However, many cancers can be cured by diagnosis and therapy in good time. There are in principle various types of therapy for, treatment of cancer. The main aim of every cancer therapy is,, however, always maximum destruction and removal of all tumor cells together with minimal damage to the normal tissue surrounding the tumor.

Localized tumorous diseases are treated mainly by local therapeutic procedures such as surgery and radio-therapy. In surgery, the primary tumor is removed as completely as possible by an operation, while the tumor cells in the primary tumor are killed by means of radiotherapy through targeted irradiation. The site of action of the irradiation is the DNA present in the cell nucleus of each cell. The irradiation leads to a large amount of DNA damage which the cell's own enzymes are unable to repair completely. As a result of this, the cell initiates programmed cell death. In further steps, the damaged cells are lysed and the fragments produced thereby are broken down by the body's immune system.

Localized tumorous diseases may, however, spread through the lymphatic system and bloodstream. Once metastases have invaded other organs of the body, local therapeutic procedures on their own are insufficient to stop further spread of the tumorous disease. In these cases, the treatment must include the whole body, and this can be achieved by chemotherapy. In chemotherapy there is targeted administration of substances, namely cytostatics, which inhibit the growth of tumor cells and thus kill the tumor cells. Known cytostatics include antimetabolites, topoisomerase inhibitors, alkylating agents and plant alkaloids. Although the effect of all cytostatics is to inhibit tumor cell growth, the fundamental principles by which the various cytostatics act differ completely. A range which is as large as possible of cytostatics with different principles of action is of crucial importance for treatment of the various tumorous diseases because each tumorous disease is unique and requires a specific type of treatment. Despite the large number of cytostatics disclosed to date, therapy of all tumorous diseases is not as yet possible. For this reason there is still a continuing interest in the development of novel cytostatics.

The present invention was therefore based on the object of producing novel complexes which are cytostatics with high antitumor activity and a broad range of actions against a large number of tumorous diseases.

This object has been achieved according to the invention by providing a complex of the general formula
D₂—M—T
where

D is a β-diketone,

M is a metal atom selected from the group consisting of Cr, Cu, Mn, Fe, Ni, Co, Zn and Mo,

T is a substance having at least one N-, O- or S-containing group, and

where M participates in an electron donor-acceptor interaction with T, and M in the complex has a free coordination site.

The complex of the invention forms a new class of monocrystalline organometallic complex compounds with tetragonal-bipyramidal geometry. The metal atom M of the complex of the invention is located in the center of the tetragonal bipyramid. The two bidentate β-diketone ligands D each occupy with their two complex-forming oxygen atoms the four equatorial positions of the tetragonal bipyramid. One of the two axial positions of the tetragonal bipyramid is occupied by substance T, with the N or O or S atom of the N- or O- or S-containing group of the substance T acting as complex-forming atom, participating in an electron donor-acceptor interaction with the metal atom M. A free coordination site is present at the other axial position of the tetragonal bipyramid. The free coordination site on the metal atom M of the complex of the invention enables a specific interaction with other molecules such as, for example, with oxygen, nitrogen oxides or a molecular binding site on the surface of the target cells etc.

It was possible to show in structural investigations that the metal atom M of the complex of the invention is about 2 Å away from its ideal position in the tetragonal bipyramid in the direction of substance T. Hence the electron donor-acceptor interaction between the metal atom M and substance T assumes the character of a double bond. It was further possible to show that two of the four equatorial positions are displaced somewhat in the direction of the metal atom, while the other two equatorial positions are disposed somewhat at a distance from the metal atom.

The β-diketone D of the complex of the invention is distinguished by its three-dimensional structure (i) enabling an optimal chelate formation with the metal atom M at its equatorial coordination sites and (ii) not disturbing the electron donor-acceptor inter-action between substance T and metal atom M. However, the β-diketone is preferably selected from the group consisting of acetylacetone and its higher alkyl analogs, dibenzoylmethane and diethyldithiocarbamine.

The metal atom M of the complex of the invention is selected from the group consisting of Cr, Cu, Mn, Fe, Ni, Co, Zn and Mo. Beyond this, the metal atom M is characterized in that it enables a tetragonal-bipyramidal arrangement of the ligands D and T, with one axial coordination site of the metal atom remaining free. Particularly preferred metal atoms M are Cu and Mn.

Substance T in the complex of the invention has at least one N-, O- or S-containing group which participates via the N or O or S atom in an electron donor-acceptor interaction with the metal atom M at one of the axial coordination sites of M. This entails the N or O or S atom of substance T acting as electron donor and providing a free electron pair to metal atom M as electron acceptor. Substance T preferably has at least one NH₂-, NH-, N-, O- or S-containing group. In a preferred embodiment of the invention, substance T itself has antitumor activity and is selected from the group consisting of 2,4-dihydroxy-5-fluoropyrimidine, 5-fluoro-1-(tetrahydro-2-furyl)uracil, 2-[bis(2-chloroethyl)amino]tetrahydro-2H-1,3,2-oxazephosphorine 2-oxide, 1,2-imidopropanoic amide (leacadine), 2-hydroxymethyl-5-hydroxy-γ-pyrone, 2,4,6-trimethylpyridine, 2,4,6-tri-2-pyridyl-1,3,5-triazine, 4-[bis(2-chloroethyl)amino]-L-phenylalanine (melphalan), 2-(3-pyridyl)piperidine, 2-2′-bipyridine, 2-methyl-(5-trimethylbutyl-1-il-ol-3)pyridine, 2-methyl-(3-dimethylamino-1-propynyl)pyridine and 2-methyl-5-ethylenepyridine.

In a preferred embodiment of the invention, the complex of the invention includes copper as central metal atom M, acetylacetone or a higher alkyl analog thereof as β-diketone D and a substance selected from the group consisting of 2,4-dihydroxy-5-fluoropyrimidine, 5-fluoro-1-(tetrahydro-2-furyl)uracil, 2-[bis(2-chloroethyl)amino]tetrahydro-2H-1,3,2-oxazephosphorine 2-oxide, 1,2-imidopropanoic amide, 2-hydroxymethyl-5-hydroxy-γ-pyrone, 2,4,6-trimethylpyridine, 2,4,6-tri-2-pyridyl-1,3,5-triazine, 4-[bis(2-chloroethyl)amino]-L-phenylalanine, 2-(3-pyridyl)piperidine, 2-2′-bipyridine, 2-methyl-(5-trimethylbutyl-1-il-ol-3)pyridine, 2-methyl-(3-dimethylamino-1-propynyl)pyridine and 2-methyl-5-ethylenepyridine as substance T.

In a further preferred embodiment of the invention, the complex of the invention includes Mn as central metal atom M, acetylacetone or its higher alkyl analogs as β-diketone D and a substance selected from the group consisting of 2,4,6-trimethylpyridine, 2,4,6-tri-2-pyridyl-1,3,5-triazine, 2-2′-bipyridine, 2-(3-pyridyl)piperidine, 1,2-imidopropanoic amide and 4-[bis(2-chloroethyl)amino]-L-phenylalanine as substance T.

It has now surprisingly emerged that the complex of the invention forms a new class of cytostatics with excellent antitumor activity. If, in a preferred embodiment of the complex of the invention, the substance T itself is an antitumor agent, it has been found that the complex of the invention shows an antitumor activity which is greatly increased compared with the substance T present therein. In addition, the complex of the invention shows immunomodulatory and antiproliferative properties plus an antiangiogenic activity, and has greatly increased hydrolysis stability compared with conventional antitumor agents, which means that it can be employed in extensive areas of tumor control. It has further been possible to establish that the complex of the invention induces no drug resistance and is able under certain conditions to bring about apoptosis and angiogenesis in cancer cells.

The complex of copper or manganese, acetylacetone and 4-[bis(2-chloroethyl)amino]-L-phenylalanine (melphalan), referred to hereinafter as MOC.melphalan, is particularly preferred for the purposes of the invention. The investigations which have been carried out show that this substance accumulates predominantly in tumor tissue, catalytically oxidizes fragments of protein receptors on the membrane surface and thus prevents metastatic processes. The substance additionally possesses immunomodulatory effects via regulation of the Tₕₑₗₚ/Tₛᵤₚᵣ ratio and influences the production of specific antibodies. The substance is chemically stable and has a sustained effect. It is particularly important that the substance overcomes the blood-brain barrier and thus makes it possible to treat brain tumors. In addition, no drug resistance is generated. Investigations of adenocarcinoma, sarcoma, leukemia cells, melanoma and renal cell carcinoma (RENCA) revealed an activity far superior to that of melphalan.

Concerning the mechanism of action of this particularly preferred substance, it is assumed that it is able to regulate the nitric oxide content and the Ca ion concentrations in tumor cells. By “sucking out” the Ca²⁺ ions from tumor cells, the glycolysis process is inhibited and thus the function of their mitochondria is impaired. In connection with the recombination of the active forms of oxygen, MOC.melphalan results in conditions which lead to destruction of the tumor cells with the assistance of macrophages—which are loaded with NO. In addition, the substance acts on gene expression through penetration into the nucleus of the tumor cells, thus destroys the cell nucleus and inhibits the proliferation activity. Finally, it has been found that it also induces a two-layer capsule formation of the tumor, with the inner layer consisting of fat-like cells. This additionally suppresses the nutrient supply to the tumor and, where appropriate, makes targeted surgical intervention possible.

A further particularly preferred substance from the complexes of the invention is the complex of copper acetylacetonate with tegafur:

Cu(C₅H₇O₂)₂.C₈H₉O₃N₂F-MOC.tegafur.

The crucial disadvantages of tegafur [5-fluoro-1-(tetrahydro-2-furyl)uracil are the short duration of action, which leads only to suppression of the synthesis of nucleic acids, and the poor solubility in water. Despite its low toxicity (LD₅₀ is 650 mg/kg), the product shows a strong effect on blood production and induces leukopenia, thrombocytopenia and anemia. It accumulates in high concentrations in brain tissue and causes diarrhea and stomatitis. The product tegafur must not be used in association with renal and hepatic diseases (in the terminal state), in association with hemorrhages and when the content of leukocytes and platelets is below 3·10⁹/l. The use of tegafur is limited to the treatment of tumors of the small and large bowel, recurrent stomach tumors, and carcinoma of the breast and ovary.

The combination according to the invention of the molecule tegafur with the copper acetylacetonate molecule leads to a novel chemical compound which does not have these numerous disadvantages. The organo-metallic complex MOC.tegafur has a wide range of antitumor-active, antimetastatic and immunoregulatory properties. It is a water-soluble substance with a prolonged action, and it induces no drug resistance in the body. A further crucial advantage is the therapeutic dosage of the product MOC.tegafur (dosage of 5 mg/kg of body weight). This is only 2.1 mg in the case of tegafur on its own. MOC-leacadine (Cu/Mn(acac)₂-leacadine) also shows the effects described in detail for MOC.melphalan, such as “sucking out” of the Ca²⁺ ions, destruction of the mitochondria in tumor cells etc.

A further aspect of the present invention accordingly relates to a pharmaceutical composition comprising at least one complex of the invention. The pharmaceutical composition may comprise a single complex of the invention or a combination of a plurality of complexes of the invention as active ingredient. The pharmaceutical composition may additionally where appropriate comprise conventionally used pharmaceutical additives sufficiently well known to the skilled person, such as, for example, physiologically tolerated carrier substances, diluents and excipients.

The pharmaceutical composition of the invention may be present in a form which can be administered topically, parenterally, intravenously, intramuscularly, subcutaneously or transdermally, and can be produced with the aid of conventional processes well known in the art. The pharmaceutical composition of the invention is preferably produced in the form of tablets or as intravenous injection or infusion.

The pharmaceutical composition of the invention is employed for the treatment of tumors. The term “tumor” as used herein includes every local increase in tissue volume as well as cells in which normal growth regulation no longer operates and uncontrolled cell division takes place. This means in the widest sense every localized swelling due to edema, acute and chronic inflammation, aneurysmatic dilation and organ swelling caused by inflammation, and in the narrowest sense a formation of new tissue (e.g. growth, blastoma, neoplasia) in the form of a spontaneous, autonomous and irreversible excessive growth, which is disinhibited to various extents, of endogenous tissue, which is usually associated with various extents of loss of specific cellular and tissue functions. Examples of tumorous diseases which can be treated with the aid of the pharmaceutical composition of the invention include bowel cancer, brain tumor, eye tumor, pancreatic carcinoma, bladder carcinoma, lung cancer, breast cancer, ovarian tumor, uterus cancer, bone tumor, gall bladder and bile duct carcinoma, head-neck tumor, skin cancer, testicular cancer, renal tumor, germ cell tumor, liver cancer, leukemia, malignant lymphoma, nerve tumor, neuroplastoma, prostate cancer, soft tissue tumor, esophageal cancer and carcinomas where the primary tumor is unknown.

The term “treatment of tumors” as used herein includes at least one of the following features: alleviation of the symptoms associated with the tumorous disease, a reduction in the extent of the tumorous disease (e.g. a reduction in tumor growth), a stabilization of the state of the tumorous disease (e.g. an inhibition of tumor growth), a prevention of further spread of the tumorous disease (e.g. a metastasis), a prevention of the occurrence or recurrence of a tumorous disease, a delaying or retardation of the progression of the tumorous disease (e.g. a reduction in tumor growth) or an improvement in the state of the tumorous disease (e.g. a reduction in tumor size).

The pharmaceutical composition of the invention is preferably administered to a patient with a tumorous disease in an amount sufficient to achieve a treatment of the corresponding tumor. The amount to be administered of the pharmaceutical composition depends in this connection on a plurality of factors such as, for example, the choice of the complex of the invention (specificity, activity etc.), the mode of administration (tablet, injection, infusion etc.), the nature and the extent of the tumorous disease and the age, weight and general condition of the patient, and can be determined straightforwardly by a person skilled in the area of tumorous disease, taking account of the abovementioned factors. However, the complexes of the invention are preferably administered in the range from 1 μg/kg of body weight of the patient to 5 mg/kg of body weight of the patient, preferably 1 μg/kg of body weight of the patient to 0.5 mg/kg of body weight of the patient and particularly preferably from 10 μg/kg of body weight of the patient to 0.1 mg/kg of body weight of the patient.

The pharmaceutical composition of the invention is administered topically, parenterally, intravenously, intramuscularly, subcutaneously or transdermally. The pharmaceutical composition is preferably administered in the form of tablets or as intravenous injection or infusion. It is also possible in a few cases for there to be targeted injection of the pharmaceutical composition into body cavities or via a catheter into the blood vessels of the tumor region or of the organ in which the tumor is located.

A further aspect of the present invention relates to the use of a complex of the invention for producing a pharmaceutical composition for the treatment of tumors.

The following examples are intended to explain the invention in more detail in conjunction with the figures in the appended drawing.

The drawings depict:

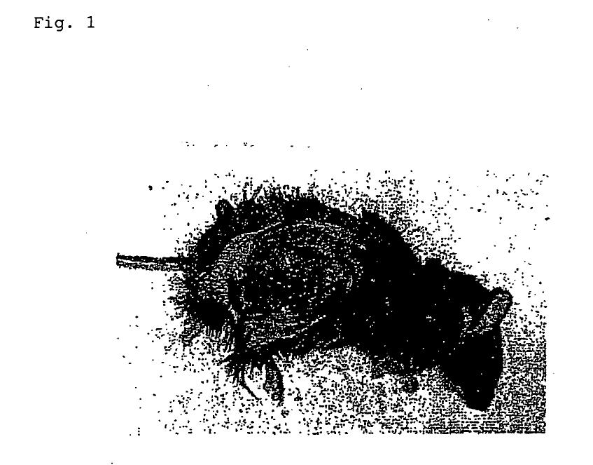

FIG. 1: black-line mouse with B-16 melanoma after treatment with MOC.melphalan, tumor weight 0.3 g

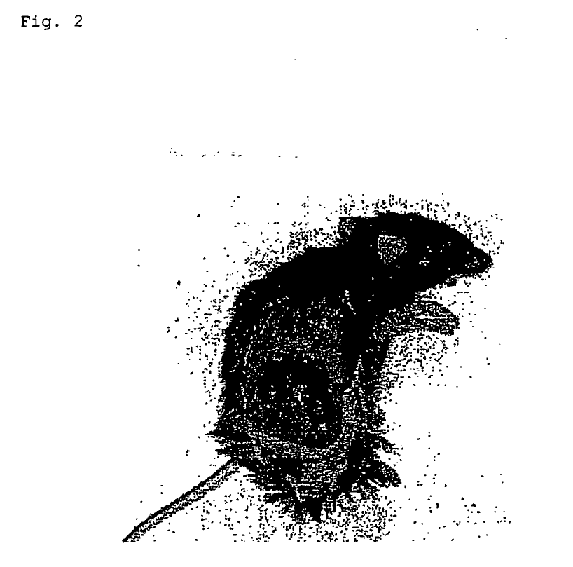

FIG. 2: black-line mouse with B-16 melanoma, control group, tumor weight 3.15 g

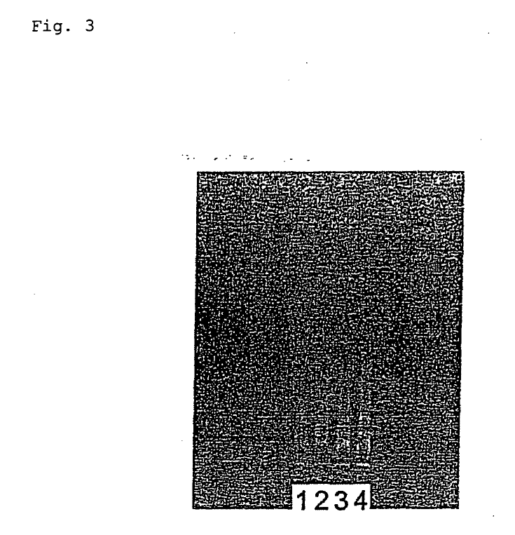

FIG. 3: DNA electrophoresis of black-line mice with B-16 melanoma
2 MOC.melphalan
4 control group
1, 3 reference substances

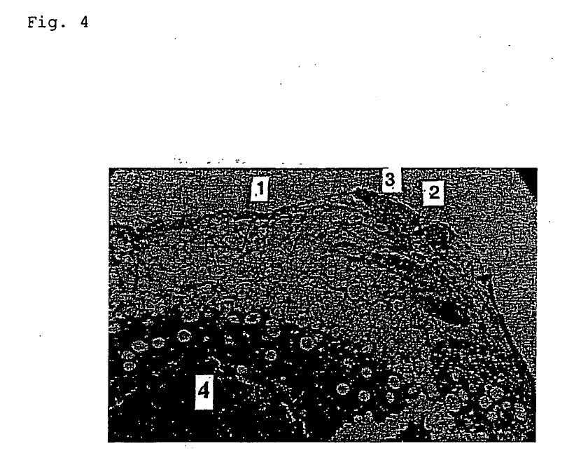

FIG. 4: effect of the substance MOC.melphalan on capsule formation of the B-16 melanoma tumor (100×magnification)
1 parenchymal fat cells
2 epithelioid cells
3 vessel
4 tumor tissue

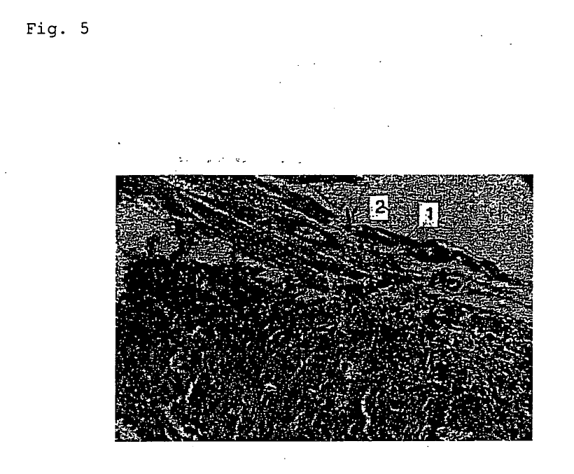

FIG. 5: B-16 melanoma tumor in the control group (100×magnification)
1 muscle fiber packages
2 vessel

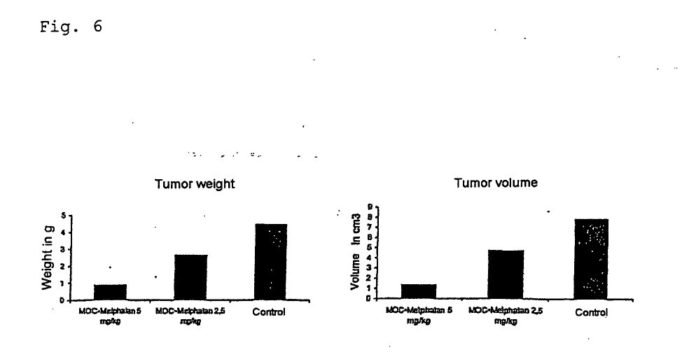

FIG. 6: shows the preventive effect of the substance MOC.melphalan on the AKATON tumor: tumor mass and volume after intravenous administration of MOC.melphalan 4 times before tumor implantation

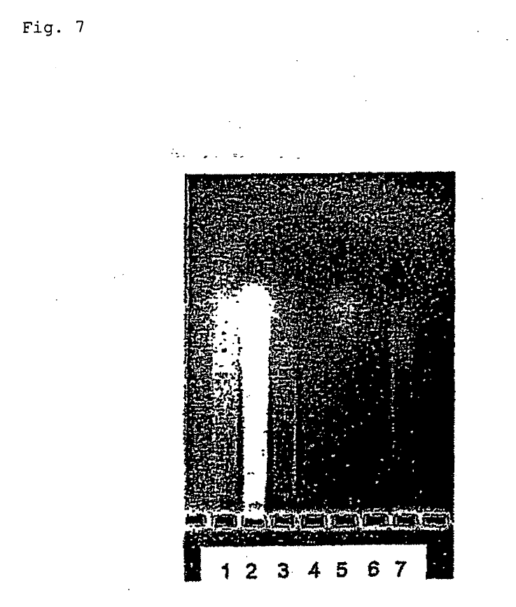

FIG. 7: shows an electropherogram of the DNA from cells of the S-180 sarcoma under the influence of the substance MOC.melphalan in a dosage of 0.05 mg/10⁵ cells
2 MOC.melphalan, incubation time 40 minutes
5 MOC.melphalan, incubation time 60 minutes
7 control group
1, 3, 4, 6 reference substances

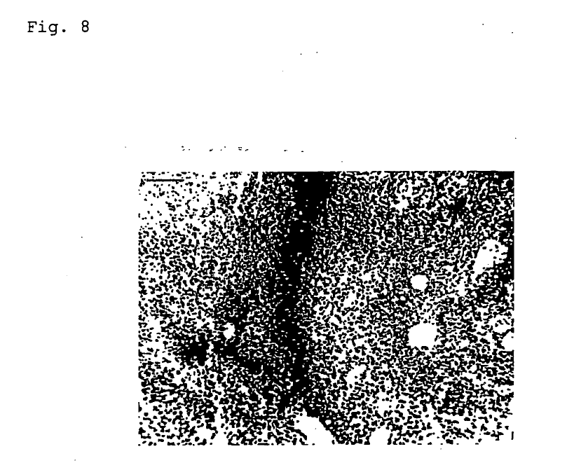

FIG. 8: formation of the two-layer sheath between malignant and healthy tissue due to the effect of the substance MOC.tegafur

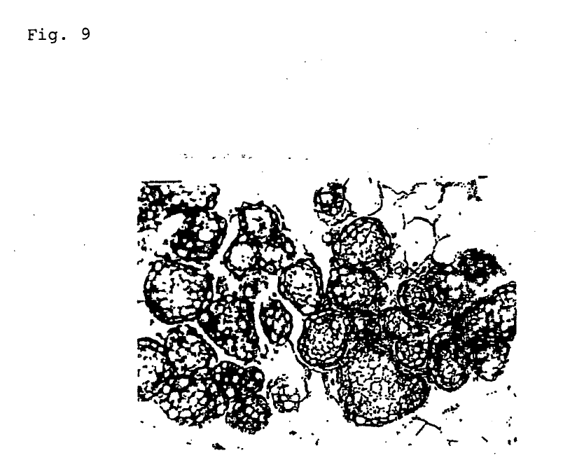

FIG. 9: formation of cavities in the cells through the action of the substance MOC.tegafur

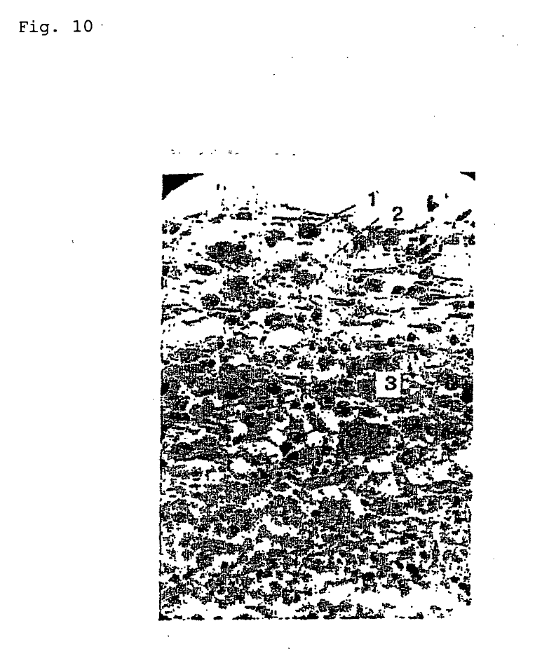

FIG. 10: effect of the substance MOC.tegafur on capsule formation of the B-16 melanoma tumor (magnification 40×40):
1 epithelioid cells with signs of degradation
2 cells of the muscle fibers
3 tumor tissue

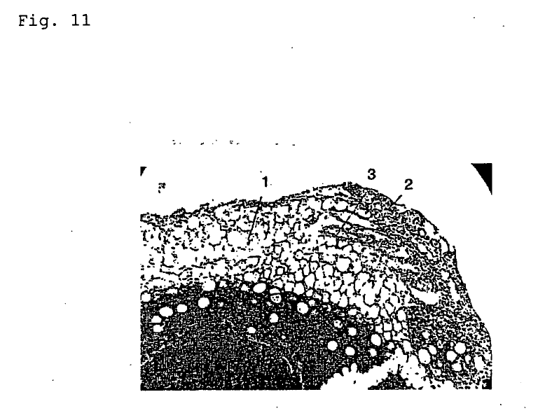

FIG. 11: effect of the substance MOC.melphalan on capsule formation of the B-16 melanoma tumor (magnification: 10×10):
1 parenchymal fat cells
2 epithelioid cells
3 vessel
4 tumor tissue

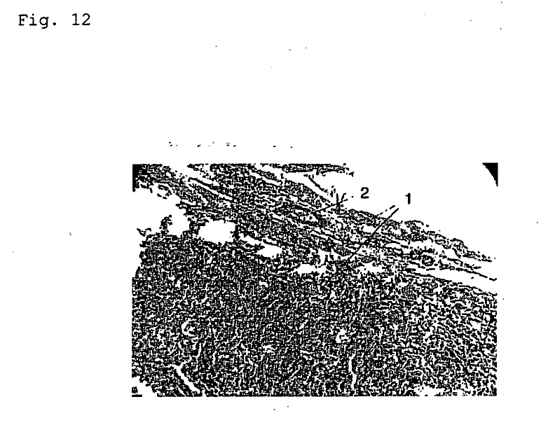

FIG. 12: B-16 melanoma tumor in animals in the control group (magnification 10×10)
1 tissue bundle of the muscles
2 vessel

### EXAMPLES

### Example 1

Preparation of the Complex of the Invention for the Example of (C₅H₇O₂)₂—Cu—C₁₃H₁₈Cl₂N₂O₂

a) Synthesis of Cu (C₅H₇O₂)₂

25 ml of freshly distilled C₅H₉O₂, dissolved in 50 ml of methanol, were added to a continuously stirred solution of 20.4 g of CuCl×2H₂O in 125 ml of water. Then a solution of 20 g of sodium acetate in 75 ml of water was added to this mixture. The mixture resulting in this way was heated to boiling in a water bath and then cooled to room temperature. The Cu(C₅H₇O₂)₂ which had formed was recrystallized from methanol. Some hours after complete crystallization, the blue Cu (C₅H₇O₂)₂ crystals were filtered off, washed with water and dried at a temperature of 80° C. in vacuo under a pressure of 6 mm Hg.

b) Synthesis of the Complex (C₅H₇O₂)₂—Cu—C₁₃H₁₈Cl₂N₂O₂

20 ml of a 0.02 molar solution of C₁₃H₁₈Cl₂N₂O₂ (4-[bis(2-chloroethyl)amino]-L-phenylalanine) were added to 0.01 mol of Cu(C₅H₇O₂)₂ in 20 ml of solvent while stirring continuously.

Variant 1: the glass vessel with the solution obtained in this way was sealed with a polyethylene closure and stored within a dark place for some days for the slow crystallization. After some days, the green (C₅H₇O₂)₂—Cu—C₁₃H₁₈Cl₂N₂O₂ crystals were removed and purified from physically adherent C₁₃H₁₈Cl₂N₂O₂ molecules using a solvent several times. The (C₅H₇O₂)₂—Cu—C₁₃H₁₈Cl₂N₂O₂ crystals were then dried in air.

Variant 2: the solution obtained in this way was evaporated in a rotary evaporator, with the solvent being drawn off under vacuum conditions (6 mm Hg) at a temperature of 40° C. The green-colored (C₅H₇O₂)₂—Cu—C₁₃H₁₈Cl₂N₂O₂ crystals were removed from the glass flask, purified with solvent and dried in air.

### Example 2

Hydrolysis Stability of the Complexes of the Invention for the Example of (C₅H₇O₂)₂—Cu—C₁₃H₁₈Cl₂N₂O₂

I. Hydrolysis Stability in Water or Physiological Saline Solution

0.6 g of (C₅H₇O₂)₂—Cu—C₁₃H₁₈Cl₂N₂O₂ was dispersed in 100 ml of water or physiological saline solution with the aid of an ultrasound generator (frequency: 15 kHz, 10 minutes). The solution obtained in this way was stable at 20° C. for a period of 30 days and showed no hydrolysis during this period.

II. Hydrolysis Stability in Olive Oil

0.6 g of (C₅H₇O₂)₂—Cu—C₁₃H₁₈Cl₂N₂O₂ was dispersed in 100 ml of 100% olive oil with the aid of an ultrasound generator (frequency: 15 kHz, 10 minutes). The solution obtained in this way was stable at 20° C. for a period of more than 2 years and showed no hydrolysis.

III. Hydrolysis Stability in Linoleic Acid or Linolenic Acid

0.1 g of (C₅H₇O₂)₂—Cu—C₁₃H₁₈Cl₂N₂O₂ was dispersed in 100 ml of linoleic acid or linolenic acid with the aid of an ultrasound generator (frequency: 15 kHz, 10 minutes). The solution obtained in this way had a pale green color and was stable at 20° C. in air for a period of 1 year.

### Example 3

Antitumor Activity of the Complexes of the Invention

1) 10 mice of the Balb line, each of which contained an adenocarcinoma, received intraabdominal administration of Cu(acac)₂M in a dose of 5 mg/kg in 0.3 ml of physiological saline solution. As control, 10 further mice of the same type with the same tumor were determined without treatment. An average tumor weight of 3.60 g was found in the control mice, while the average tumor weight in the treated mice was 0.3 g. The results for the mice treated according to the invention are indicated, with weight and size of the tumor, in table 1 below. Compared with the control series, a 91.9% inhibition of the tumor emerges therefrom.

TABLE 1

| | weight of the tumor in | Size of the tumor |
|---|---|---|
| Mice | g | cm |
| 1 | 0 | 0 |
| 2 | 0 | 0 |
| 3 | 0.35 | 0.2 × 0.2 × 0.2 |
| 4 | 1.13 | 1.4 × 0.7 × 0.6 |
| 5 | 0.8 | 0.8 × 0.8 × 0.5 |
| 6 | 0 | 0 |
| 7 | 0 | 0 |
| 8 | 0 | 0 |
| 9 | 0.49 | 0.3 × 0.2 × 0.2 |
| 10 | died | — |

Table 2 below shows the results of the control series of untreated mice, likewise indicating the weight and size of the tumor. The average weight of the tumor was 3.60 g.

TABLE 2

| | weight of the tumor in | Size of the tumor |
|---|---|---|
| Mice | g | cm |
| 1 | 1.61 | 1.6 × 1.0 × 0.7 |
| 2 | 1.69 | 2.5 × 2.0 × 0.8 |
| 3 | 6.48 | 3.6 × 2.5 × 1.6 |
| 4 | 2.93 | 3.0 × 1.0 × 0.7 |
| 5 | 3.75 | 3.5 × 1.5 × 0.7 |
| 6 | 2.56 | 3.1 × 1.0 × 1.7 |
| 7 | 3.69 | 3.0 × 2.0 × 1.0 |
| 8 | 5.22 | 3.0 × 2.5 × 1.0 |
| 9 | 4.85 | 3.0 × 2.0 × 1.0 |
| 10 | 3.23 | 2.6 × 1.5 × 1.0 |

2. Antitumor Activity for Adenocarcinoma and Intra-Venous Administration (Four Times in Physiological Saline Solution)

The results are shown in Table 3 below.

TABLE 3

| | Dose of the | Number of | Mass of the | % |
|---|---|---|---|---|
| Products | product | animals | tumor g | inhibition |
| (Cu(acac)₂M | 5 mg/kg | 6 | 0.9 | 80 |
| Control | — | 6 | 4.4 | |

3. Antitumor Activity for Adenocarcinoma Transplanted After Administration of the Product.

TABLE 4

| | | | | | Size of | |
|---|---|---|---|---|---|---|
| | Dose of | Number | Mass | | the | % |
| | the | of | of the | | tumor | inhi- |
| Products | product | animals | tumor g | % inhibition | in cm³ | bition |
| Cu(acac)₂M | 5 mg/kg | 6 | 0.91 | 80 | 1.38 | 82.5 |
| Control | | 6 | 4.46 | | 7.9 | |

The active ingredient of the invention was administered intravenously in the dosage stated in the table 4 days in succession in physiological saline solution of 0.3 ml each time. The adenocarcinoma was then implanted in the Balb mice. 21 days after the tumor was transplanted, the animals were sacrificed and the weight and size of the tumor recorded. The animals received no other drugs during the experiment and were kept with a normal feed ration. The results show that the active ingredient of the invention can accumulate in the body and has a prolonged effect.

4. Antitumor Activity for C-180 Sarcoma

The results of series of experiments in which the stated active ingredient was administered intra-peritoneally in the stated dosage in physiological saline solution are shown in table 5 below.

TABLE 5

| | Dose of the | Number of | Mass of the | % |
|---|---|---|---|---|
| Product | product | animals | tumor g | inhibition |
| Cu(acac)₂M | 5 mg/kg | 6 | no tumor | 100 |
| Melfalan | 5 mg/kg | 10 | 2.4 ± 1.1 | 49 |
| Control | | 10 | 2.8 | |

5. Efficacy for Leukemia

The therapeutic efficacy was investigated on the leukemia tumor strains L-1210, P-388, and P-388 strains specifically obtained for drug resistance. The lifespan of the animals was set at 60 days in this case. Drug-resistant tumor were obtained by successive transplantation of the leukemia P-388 with ascites cells taken from mice which had been treated with rubomycin (strain P388/ph), vincristine (P388/vcr) and zisplatin (P388/cPt).

Resistance to the products mentioned appear in the 8th, 6th and 4th generation. The investigations revealed that the P388/ph and P388/vcr strains had a phenotype and a genotype with multifactorial drug resistance.

The results are shown in tables 6, 7 and 8 below.

5a. Leukemia L-1210

Inoculum: 10⁶ cells in 0.2 ml of physiological saline solution. Mice: BDF₁, females 19-21 g. The products were administered intraabdominally.

After transplantation of the tumor, the substance MOC.melphalan was administered intraabdominally to the animals in a dosage of 5 mg/kg in 0.3 ml of 10% Twin-80 solution on days 1 to 7. The effect of the substance was assessed on the basis of the lifespan and the weight change of the animals (table 9). The observation period was 60 days.

TABLE 6

| | | | Number | | | Lifespan |
|---|---|---|---|---|---|---|
| | | | of | | | of the |
| | Single | Administration | animals | % of | Change | animals in |
| | dose | regime | in the | surviving | in | the experiment |
| Product | mg/kg | days | experiment | animals | weight g | (days) |
| Cu(acac)₂M | 5 | 1-7 | 6 | 100 | −1.5 | >60 |
| Control | 5 | 1-7 | 6 | 0 | +0.7 | 8.5 |

5b. Leukemia P-388

Inoculum: 10⁶ cells in 0.2 ml of physiological saline solution. Mice: BDF₁. Females 19-21 g

After transplantation of the tumor, the substance MOC.melphalan was administered intraabdominally to the animals in a dosage of 5 mg/kg in 0.3 ml of 10% Twin-80 solution on days 1 to 7. The effect of the substance was assessed on the basis of the lifespan and the weight change of the animals (table 7).

TABLE 7

| | | | | Number of | | | |
|---|---|---|---|---|---|---|---|
| | | | Number of | animals | | Increase | |
| | Single | | animals in | which | Average | in | |
| | dose | Administration | the | survived to | lifespan, | average | Change in |
| Product | mg/kg | regime days | experiment | day 60 | days | lifespan % | weight in g |
| Cu(acac)₂M | 5 | 1-7 | 6 | 0 | 16.8 | 56.0 | −2.5 |
| Control | | | 6 | 0 | 10.8 | — | +1.6 |

The products were administered intraabdominally. Cu(acac)₂M was dissolved in 10% Twin 80.

TABLE 8

| | | | | | Number of |
|---|---|---|---|---|---|
| | | | | | surviving |
| | | | Regime | | animals/ |
| | | | (days | | number of |
| | | Dose | after | | animals in |
| Strain | Product | mg/kg | transplantation) | ILS %* | group |
| P-388** | Cu(acac)₂M | 5 | 1-7 | 56 | — |
| o.S. | | 10 | 1, 5, 9 | 419 | 5/6 |
| (initial | | 10 | 1, 7 | 465 | 4/6 |
| strain) | | 15 | 1, 7 | 447 | 5/6 |

Drug-resistant tumors

| | | | | | |
|---|---|---|---|---|---|
| P388/ph | Cu(acac)₂M | 5 | 1-7 | 189 | 2/6 |
| P388/vcr | | 5 | 1-7 | 516 | 5/6 |
| P388/cPt | | 5 | 1-7 | 193 | — |

*Average percentage survival time In the ILS determination, the reference value for the survival rate of the animals was fixed at 60 days.
**o.S.—original strain, not resistant
pH—rubomycin-resistant strain
vcr—vincristine-resistant strain
cPt—cisplatin-resistant strain

The drug-resistant tumors were obtained by administration of ascites cells leukemia P-388 which were derived from mice treated with rubomycin, vincristine and cisplatin. The resistance was found in the 4th, 6th and 8th generation. The sensitivity of the resistant tumors was reduced 4-5-fold through the use of the substance MOC″melphala.

As is evident from tables 6 to 8, the most interesting results were obtained with leukemia P-388 with multifactorial drug resistance. These tumors, which respond only weakly to numerous antitumor agents, were sensitive to the product of the invention.

The effect on L-1210 is equally remarkable, because all the animals in the experimental group survived for 60 days after transplantation of the tumor. Such a survival time corresponds to complete cure thereof.

### Example 4

Immunomodulatory Properties of the Complexes of the Invention for the Example of (C₅H₇O₂)₂—Cu—C₁₃H₁₈Cl₂N₂O₂

The immunomodulatory properties of the complex (C₅H₇O₂)₂—Cu—C₁₃H₁₈Cl₂N₂O₂ of the invention were determined on the basis of the increase in antibody-forming cells of white, crossbred mice (average weight: 20 g). The mice were immunized intraperitoneally with 2×10⁸ sheep erythrocytes in 0.2 ml of physiological saline solution. Half an hour after the immunization, the mice received 0.3 mg/kg (C₅H₇O₂)₂—Cu—C₁₃H₁₈Cl₂N₂O₂ in 0.6 ml of olive oil by oral administration. After 4 days, the animals were sacrificed, the spleen was removed and suspended homogeneously in solvent, and 0.5 ml of the suspension was streaked on an agar in a Petri dish with sheep erythrocytes. The experiments showed that (C₅H₇O₂)₂—Cu—C₁₃H₁₈Cl₂N₂O₂ in a dosage of 0.3 mg/kg of body weight increased the number of antibody-forming cells in the spleen of immunized animals. Thus, the number of antibody-forming cells in the control group, which received only 0.6 ml of olive oil by oral administration, was 76 000±5 000, whereas the number of antibody-forming cells in the immunized animals was 158 000±7 000.

### Example 5

Toxicity Investigations on the Complexes of the Invention for the Example of (C₅H₇O₂)₂—Cu—C₁₃H₁₈Cl₂N₂O₂

The toxicity of the complex (C₅H₇O₂)₂—Cu—C₁₃H₁₈Cl₂N₂O₂ of the invention was determined on five laboratory species in various tests. The results of the investigations showed that (C₅H₇O₂)₂—Cu—C₁₃H₁₈Cl₂N₂O₂ in a dosage of 5 mg/kg of body weight causes no serious changes in the peripheral blood count, has no pathological effects on renal and hepatic function, and causes no specific changes in organs and tissues. It was additionally possible to show in the investigations that (C₅H₇O₂)₂—Cu—C₁₃H₁₈Cl₂N₂O₂ induces no resistance even on prolonged administration.

### Example 6

Antitumor Activity for B-16 Melanoma

Experiment I

After transplantation of the B-16 melanoma tumor to Black-line mice. (cell suspension with 10×10⁶ cells/ml), MOC.melphalan was administered intraabdominally in a dosage of 5 mg/kg in 0.3 ml of 10% DMSO solution on days 3, 5 and 9. The animals were sacrificed on day 21 of the investigation and underwent histological, morphological examination (see table 9).

TABLE 9
Effect of the substance MOC.melphalan on B 16 melanoma tumor
Inhibition of tumor proliferation

| | | | Average | |
|---|---|---|---|---|
| | Dosage in | Number of | tumor | Percentage |
| Group | mg/kg | animals | mass/g | inhibition |
| MOC.melphalan | 5 | 5 | — | 100 |
| Control | — | 5 | 3.4 | — |

Experiment II

After transplantation of the B-16 melanoma tumor into black-line mice (cell suspension with 10×10⁶ cells/ml), the substance was administered intraabdominally in a dosage of 0.1 mg/animal in 0.3 ml of 10% DMSO solution on days 3, 5 and 9. The animals were sacrificed on day 16 of the investigation and underwent histological, morphological examination (see table 10, FIGS. 1, 2). The DNA concentration in the tumor cells was determined by spectrophotometry after electrophoresis. (Table 10, FIG. 3).

TABLE 10
Effect of the substance MOC·melphalan on the B-16 melanoma tumor
Inhibition of tumor proliferation, destruction of tumor cell DNA

| | Dosage | Number | Average | | | |
|---|---|---|---|---|---|---|
| | mg/ | of | tumor | Percentage | DNA | Mitosis |
| Group | animal | animals | mass/g | inhibition | mg/g | index |
| MOC· | 0.1 | 6 | 0.83 | 78 | 0.8 | 0.6 |
| melphalan | | | | | | |
| Control | — | 6 | 3.22 | — | 4.3 | 4.2 |

### Example 7

Activity for S-180 Sarcoma

Experiment I

After transplantation of the S-180 sarcoma tumor into white crossbred mice, the animals received the substance MOC.melphalan by intraabdominal administration in a dosage of 1 mg/kg in 0.3 ml of 10% DMSO solution on days 3, 5 and 9. The animals were sacrificed on day 21 of the investigation and underwent histological, morphological examination (table 11).

TABLE 11
Effect of the substance MOC.melphalan on the S-180
sarcoma tumor
Inhibition of tumor proliferation

| | | | Average | |
|---|---|---|---|---|
| | Dosage in | Number of | tumor | Percentage |
| Group | mg/kg | animals | mass/g | inhibition |
| MOC.melphalan | 1 | 6 | — | 100 |
| Control | — | 6 | 4.7 | — |

Experiment II

After transplantation of the S-180 sarcoma tumor into white crossbred mice, the animals received MOC.melphalan and melphalan by intraabdominal administration in a dosage of 5 mg/kg in 0.3 ml of 10% DMSO solution on days 3, 5, 7 and 9. The animals were sacrificed on day 21 of the investigation and underwent histological, morphological examination (table 12).

TABLE 12
Effect of the substance MOC.melphalan on the S-180
sarcoma tumor
Inhibition of tumor proliferation

| | | | Average | |
|---|---|---|---|---|
| | Dosage in | Number of | tumor | Percentage |
| Group | mg/kg | animals | mass/g | inhibition |
| MOC.melphalan | 5 | 6 | — | 100 |
| Melphalan | 5 | 10 | 1.4 | 49 |
| Control | — | 10 | 2.8 | — |

Experiment III

After transplantation of the S-180 sarcoma tumor into white crossbred mice, the animals received MOC.melphalan and melphalan by intraabdominal administration in a dosage of 5 mg/kg in 0.3 ml of 10% DMSO solution on days 2, 4, 6, 8 and 10. The animals were sacrificed on day 21 of the investigation and underwent histological, morphological examination (table 13).

TABLE 13
Effect of the substance MOC.melphalan on the S-180
sarcoma tumor
Inhibition of tumor proliferation

| | | | Average | |
|---|---|---|---|---|
| | Dosage in | Number of | tumor | Percentage |
| Group | mg/kg | animals | mass/g | inhibition |
| MOC.melphalan | 5 | 10 | 2.0 ± 0.7 | 49 |
| Melphalan | 5 | 10 | 2.4 ± 1.1 | 38 |
| Control | — | 20 | 3.9 ± 0.5 | — |

### Example 8

Effect on Small Bowel Adenocarcinoma (AKATON)

Experiment I

After transplantation of the AKATON tumor into white crossbred mice, the animals received the substance MOC.melphalan by intraabdominal administration in a dosage of 5 mg/kg in 0.3 ml of physiological solution on days 3, 5, 7 and 9. The animals were sacrificed on day 21 of the investigation and underwent histological, morphological examination (table 14).

TABLE 14
Effect of the substance MOC.melphalan on the AKATON tumor
Inhibition of tumor proliferation on intraabdominal administration

| | | | Average | |
|---|---|---|---|---|
| | Dosage in | Number of | tumor | Percentage |
| Group | mg/kg | animals | mass | inhibition |
| MOC.melphalan | 5 | 10 | 0.3 | 92 |
| Control | — | 10 | 3.6 | — |

Experiment II

After transplantation of the AKATON tumor into white crossbred mice, the animals received the substance MOC.melphalan by intravenous administration in a dosage of 5 mg/kg in 0.3 ml of physiological solution on days 3, 5, 7 and 9. The animals were sacrificed on day 21 of the investigation and underwent histological, morphological examination (table 15).

TABLE 15
Effect of the substance MOC.melphalan on the AKATON tumor
Inhibition of tumor proliferation on intravenous administration

| | | | Average | |
|---|---|---|---|---|
| | Dosage in | Number of | tumor | Percentage |
| Group | mg/kg | animals | mass/g | inhibition |
| MOC.melphalan | 5 | 6 | 0.9 | 80 |
| Control | — | 6 | 4.4 | — |

Experiment III

Investigation of tumor prevention by the substance MOC.melphalan

Mice of the Balb line received the substance MOC.melphalan in dosages of 2.5 mg/kg and 5 mg/kg in 0.3 ml of physiological solution by intravenous administration on four consecutive days (1× a day). On day 5, the AKATON tumor was implanted in the animals. The animals were sacrificed on day 21 of the investigation and underwent histological, morphological examination (table 16, FIG. 6).

TABLE 16
Preventive effect of the substance MOC·melphalan on the
AKATON tumor

| | | | | Percent | | Percent |
|---|---|---|---|---|---|---|
| | | | | inhibition | Average | inhibition |
| | | Number | Average | of | tumor | of |
| | Dosage | of | tumor | tumor | volume/ | tumor |
| Group | mg/kg | animals | mass/g | weight | cm³ | volume |
| MOC· | 5.0 | 6 | 0.91 | 80 | 1.38 | 82 |
| melphalan | | | | | | |
| MOC· | 2.5 | 6 | 2.67 | 40 | 4.76 | 40 |
| melphalan | | | | | | |
| Control | — | 6 | 4.46 | — | 7.90 | — |

— inhibition of tumor proliferation

### Example 9

Determination of the DNA Concentration in Cells from Mice with S-180 Sarcoma after the Action of the Substance MOC.Melphalan

S-180 sarcoma tumor cells (20·10⁶ cells/investigation) were incubated with the substance MOC.melphalan in a concentration of 0.05 mg/10⁶ cells at a temperature of 37° C. for 40 or 60 min (see table 16).

The DNA concentration was determined using the phenol-chloroform method of MANIATIS (Maniatis, Frin, Sämbruck Methods of genetic engineering, Molecular Cloning, M.: MIR, 1984, pages 479 et seq.). Separation of DNA and RNA was followed by a phoresis in a 2.5% agarose gel (see table 16, FIG. 7). The amount of DNA was calculated from the RN-ase consumption, i.e. the data indicate the exact mass of DNA.

TABLE 17
Effect of the substance MOC.melphalan on the DNA
concentration in S-180 sarcoma tumor cells

| | | dosage | DNA | DNA |
|---|---|---|---|---|
| | Incubation | mg/10⁶ | concentration | concentration/ |
| Group | time/min | cells | μg/g of cells | control |
| MOC.melphalan | 40 | 0.05 | 530 | 498 |
| MOC.melphalan | 60 | 0.05 | 53 | 50 |
| Control | — | — | 106 | 100 |

### Example 10

Synthesis of the Cu(acac)₂.Tegafur Complex

The Cu(acac)₂.tegafur complex was obtained by slow crystallization from a chloroform/methanol solution acidified with hydrochloric acid.

Preparation: 2.61 g (1×10⁻mol) of copper acetyl-acetonate Cu(C₅H₇O₂) are dissolved in 50 ml of purified chloroform. The solution has a dark blue color. 4 g (2×10⁻² mol) of tegafur (N¹-(2-furanidil)-5-flururacil) are dissolved in 50 ml of chloroform/methanol solution in the ratio 1:1. The resulting solutions were heated to boiling in a water bath and mixed by continuous stirring with the aid of a magnetic stirrer. The solution assumes a brilliant green color. The glass vessel with the solution is put in a dark place for slow crystallization. 3 to 4 days after complete evaporation of the solvent, the brownish green residue is purified with chloroform until all the unreacted Cu(C₅H₇O₂)₂ and tegafur have been washed out.

The remaining green crystals are dried in air.

Composition and structure: of the MOC.tegafur complex were carried out by the methods of EPR, NMR, electron spectroscopy and infrared spectroscopy.

Characterization of the Compound MOC.Tegafur

The product is a polycrystalline organometallic complex having a green color. The stoichiometric Cu(C₅H₇O₂)₂:tegafur ratio is 1:1. The molecular mass of the Cu(acac)₂.tegafur complex is 461.2 g/mol, and the complex is readily soluble in water, in physiological solution, in methanol, ethanol, DMSO and Twin-80. The compound is insoluble in ether and chloroform. The melting point of the crystals is 127° C. The compound is stable in air for more than 5 years.

Spectroscopic Parameters of MOC.Tegafur

UV spectra (solution: methanol/chloroform, ratio 1:3): UV-VIS spectrum:
UV range: intensity lines at 36 200 cm⁻¹ (276 nm)
VIS range: characteristic transition from dₓ²_{-y}²→d_{xy} at λ=12 345 cm⁻¹ (810 nm)

ESR spectra: methanol/chloroform solution in the ratio 1:3 at room temperature and temperature of 77° K:
g_{II}=2.301, g_{⊥}=2.05, g₀=2.146
A_{II}=160·10⁻⁴ cm⁻¹, A_{⊥}=14·10⁻⁴ cm⁻¹, A₀=52·10⁻⁴ cm⁻¹

NMR spectra in deuterated methanol/chloroform (1:3): the values for the chemical shift of the protons in the NMR spectrum of the MOC.tegafur complex and of pure tegafur are compared in the tables (a, b, c, d, f correspond to the proton position).
MOC.Tegafur:

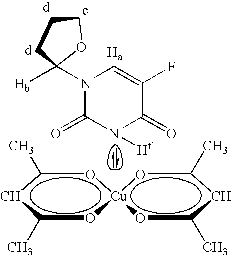

Values of the chemical shift of the protons in the substances tegafur and MOC.tegafur

TABLE 18

| Substance | a | b | c | g | f |
|---|---|---|---|---|---|
| Tegafur | 7.8 | 5.8 | 3.7; 4.1 | 2.0 | — |
| MOC·tegafur | 7.82 | 5.86 | 4.15 | 1.9 | 11.73 |

### Example 11

Antitumor Effect of MOC.Tegafur

Experiment I

48 hours after transplantation of the AKATON tumor (adenocarcinoma of the small bowel), the mice of the Balb line received the substance MOC.tegafur in a dosage of 5 mg/kg of body weight by intraabdominal administration in 0.3 ml of a physiological solution. This took place on days 3, 5, 7 and 9. The animals were sacrificed on day 21 of the investigation.

TABLE 19
Effect of the substances MOC.tegafur and tegafur on the
AKATON tumor (administration of the substances four times)

| | | Number | Tumor | |
|---|---|---|---|---|
| | Dose | of | weight | Percent |
| | (mg/kg) | animals | (g) | inhibition* |
| MOC.tegafur | 5 | 10 | 0.61 | 86 |
| Tegafur | 250 | 10 | 1.95 | 54.5 |
| Tumor control | — | 10 | 4.4 | — |

*Compared with the control group

Experiment II:

48 hours after transplantation of the AKATON tumor, the mice of the Balb line received the substance MOC.tegafur by intravenous administration in 0.3 ml of a physiological solution. This took place on days 3, 5, 7 and 9. The animals were sacrificed on day 21 of the investigation.

TABLE 20
Effect of the substances MOC.tegafur and tegafur on the
AKATON tumor on intravenous administration (administration
of the substances four times)

| | | Number | Tumor | |
|---|---|---|---|---|
| | Dose | of | weight | Percent |
| | (mg/kg) | animals | (g) | inhibition* |
| MOC.tegafur | 5 | 10 | 0.86 | 80 |
| Tegafur | 250 | 10 | 2.1 | 51 |
| Tumor control | — | 10 | 4.3 | — |

*Compared with the control group

Experiment III:

48 hours after transplantation of the sarcoma-180 tumor, mice of the Balb line received the substance MOC.tegafur by intraabdominal administration in 0.3 ml of a physiological solution. Administration took place on days 3, 5 and 9. The animals were sacrificed on day 21 of the investigation.

TABLE 21
Effect of the substances MOC.tegafur and tegafur on the
sarcoma-180 tumor (administration of the substances three times)

| | | | Tumor | |
|---|---|---|---|---|
| | Dose | Number of | weight | Percent |
| | (mg/kg) | animals | (g) | inhibition* |
| MOC.tegafur | 3 | 6 | 0.43 | 89 |
| MOC.tegafur | 5 | 6 | 0.195 | 95 |
| Tegafur | 250 | 10 | 3.1 | 20.5 |
| Control | — | 6 | 3.9 | — |

*Compared with the control group

### Example 11

Investigation of the Effect of MOC.Tegafur on AKATON Tumor Proliferation After Transplantation of the Tumor

The product was administered intravenously in a physiological solution of 0.3 ml on 4 days in succession (table 20). The AKATON tumor was then transplanted.

TABLE 22
Effect of MOC·tegafur on tumor proliferation in the
investigations on AKATON

| | | | | Percent | | Percent |
|---|---|---|---|---|---|---|
| | | | | inhibition | Size of | inhibition |
| | | Number | Tumor | of | the | of |
| | Dose | of | weight | tumor | tumor | tumor |
| | (mg/kg) | animals | (g) | weight | (cm³) | volume |
| MOC· | 5 | 6 | 2.20 | 50* | 3.06 | 61.3* |
| tegafur | | | | | | |
| Control | — | 6 | 4.46 | — | 7.90 | — |

*Compared with the control group

### Example 12

Structural Morphological Changes in Tumor Tissue in Mice with Sarcoma-180 Through the Action of MOC.Tegafur

The sarcoma-180 tumor was transplanted with a weight of 20 to 22 g into sexually mature crossbred mice. 48 hours after the transplantation of the sarcoma-180 tumor, the mice received the substance MOC.tegafur in a dosage of 5 mg/kg of body weight by intraabdominal administration in 0.9% NaCl solution. Administration took place on days 3, 5 and 9.

On day 21, the mice were sacrificed by decapitation and the tumor tissue was removed for histological examination. The antitumor activity of MOC.tegafur was provisionally determined. The percent inhibition of tumor proliferation in this investigation was 96.4%.

The morphological structure of the histological tumor sections in the animals which received MOC.tegafur differed from the tumors of the animals in the control group. Firstly, a two-layer sheath, comparable to a capsule, forms between the healthy and tumor tissues.

It consists of muscle fibers on the outside, and cells with membranes can be identified on the inside. Most of the cells have no contents (FIG. 8). Secondly, a large number of cavities of various sizes comparable with vacuoles is evident (FIG. 9).

Highly differentiated cells and necrotic and post-necrotic zones are to be seen in tumor tissue of the animals treated with MOC, as are blood vessels which contain a large number of cells. The tumor cells are usually round with small nuclei (one or two nuclei) and contain micronuclear, micro-crosslinked, diffusely distributed chromatin. A few of the large blast cells appear round. The cytoplasm which surrounds the large nucleus is not uniformly distributed. The numerous, embracing chromatin is concentrated at the periphery of the nucleus. The number of multinuclear cells indicates an impairment of cytokinesis. Early condensation of the chromatin is to be seen in the blast-transformed cells.

In the investigation, the activity of the tumor tissue was determined in animals which received the product MOC.tegafur and in untreated animals in the control. The number of mitoses and the mitosis index was calculated. The average number of mitoses in the investigation group was 2.55% and in the control animal group was 11.2%. The mitosis index in these animal groups was 0.9 and 4.75. On examination under the microscope, an increase in the number of pathological metaphases and anaphases was observed.

In summary, it is evident from this that the product MOC.tegafur induces a great destructive change in tumor tissue.

### Example 13

Effect of MOC.Melphalan and MOC.Tegafur on the B-16 Melanoma Tumor

48 hours after transplantation of the B-16 melanoma tumor in mice of the Black line, the substances MOC.melphalan and MOC.tegafur were administered intraabdominally in the dosage of 3 mg/kg of body weight (in the following on days: 3, 5 and 9). Taking account of the poor solubility of MOC.melphalan in water, it was dissolved in 10% DMSO solution (made up with 0.15 molar NaCl). MOC.tegafur was administered in a physiological solution. The animals in the control group likewise received administration of the solutions (without products). On day 16, the mice were sacrificed by decapitation, tumor tissue was removed for histological examination, and the tumor was investigated for antitumor activity (table 23). The tumors were fixed with 10% formalin, which was followed by embedding in paraffin. The section thickness of 5 μm were stained with hematoxylin-eosinome. The microscopy was carried out with a Leica Galen microscope.

The results of investigations on these compounds, which are compiled in table 23, indicate a high antitumor activity.

TABLE 23

| | | | | | MI |
|---|---|---|---|---|---|
| | Number of | Dose | Tumor | Percent | (mitosis |
| | animals | mg/kg | weight g | inhibition* | index) |
| MOC·tegafur | 5 | 5.0 | 1.13 | 65.66 | 0.86 |
| MOC·melphalan | 5 | 5 | 0.74 | 77.71 | 0.64 |
| Control | 5 | | 3.22 | | 4.22 |

*compared with the control group

When exposed to the tumor, the substances act in the logarithmic proliferation stage not only on the size and the weight of the tumors; on the contrary, they additionally inhibit the processes of division and the viability of the tumor cells.

An inhibition of tumor proliferation was observed in the groups of animals which received the substances MOC.melphalan and MOC.tegafur, but the number of mitotic cells was also lower (table 21).

These particular actions of the compounds are clearly evident in the morphology. A capsule formation is to be observed in the microscopy of the animals' tumors (the animals which received MOC.tegafur (FIG. 10)). Various cell types are evident inside the capsules: firstly epithelioid cells with degradation and secondly muscle cells. Tumor tissue is also evident. A large number of zones of necrosis is to be observed in the tumor tissue. The tumor cells are highly differentiated, with different cell nuclei. The chromatin is mostly macronuclear, micro-crosslinked, and cells with much chromatin can also be seen. The number of divisible cells is low (MI is 0.86).

A different picture of the morphological structure is to be seen in the tumor tissue of the animals which received MOC.melphalan (FIG. 11). The upper layer of the capsule is in this case considerably larger, more uniform and consists of parenchymal fat cells. Cells of the same type are evident at the front boundary of the tumor tissue. Kerotic sections and stripes are to be seen in the center of the tumor. The destruction can also be seen in the intercellular contacts. The tumor cells mostly have little cytoplasm, and the nuclei are distorted and contain a granular chromatin. The number of divisible cells is low. A small number with large transformed nuclei is evident. Alveolar structures are also present in the tumor tissue and are formed from the antitypical melanoblastoma-epithelioid types.

In the animal tumors in the control group there is no capsule formation (FIG. 12). The outer layer consists of muscle bundles which are present in most cases in the tumor tissue. Most of the actively divisible tumor cells show predominantly a normal progress of mitosis. The nuclei are large and contain various types of chromatin. Small necrotic cells are rare.

Comparative analysis of the morphological appearances of the three animal groups leads to the conclusion that the complexes of the invention have a targeted effect on tumor tissues. Despite the differences in the morphological structure, the trend of the effect of the products on tumor degradation is equal to the following: encapsulation, substantial degradation of chromatin, tissue disintegration, reduction in proliferative cell activity.

### Example 14

Effect on Cell Cultures

The effect of the substances MOC.melphalan and MOC.tegafur of the invention on the proliferative activity, morphology and protein synthesis was investigated on tumor cell cultures (KML). This took place with the mice separated out of the melanoma-16 line. 80 000 cells/ml were distributed in 3 ml of DMEM with the addition of embryonic calf serum, 200 μ/mol of glutamine and antibiotic. After cultivation of the cells at a temperature of 37° C. for 24 hours (logarithmic cell growth phase), the MOC substances were added in the concentration of 10 and 100 μg/ml. After incubation for 24 hours, the number of live cells was determined. In parallel, the fixed specimens of the cells were prepared for morphological analysis.

Both MOC substances in the concentration of 100 μg/ml cause suppression of tumor growth (table 24) and a further death of tumor cells.

TABLE 24
Investigations on the suppression of cell proliferation
through the action of MOC.tegafur and MOC.melphalan

| | | Suppression* of |
|---|---|---|
| | Dose (μg/ml) | growth cells (%) |
| MOC.tegafur | 100 | 100 |
| | 10 | 32 |
| MOC.melphalan | 100 | 95 |
| | 10 | 30 |

*compared with the tumor control

Morphological analysis showed that at the dosage of 10 μg/ml MOC.tegafur causes an accumulation of hyperchromic and pycnotic cells. MOC.melphalan leads to cell formation with cytoplasm, similar to a vacuole.

The effect of the substances on protein synthesis is determined with the aid of the suppression stage through the inclusion of radioactive precursors of amino acids (totaled mixture of ³H-amino acids). 1 million cells in a “log phase” were selected for this. The complexes were introduced in a dosage of 50 μg/ml and, in parallel, the amino acids with an activity of 10 mCᵢ/ml were introduced into 10 ml bottles. After incubation for 24 hours, the cells were washed out from the cultivation nutrient medium, and a lysis (disruption) was carried out. The radioactivity of the proteins was determined in a cell counter (table 25).

TABLE 25
Effect of MOC.tegafur and MOC.tegafur on protein biosynthesis

| | | Suppression* of |
|---|---|---|
| | | amino acid |
| | Dose μg/ml) | inclusion (%) |
| MOC.tegafur | 50 | 77 |
| MOC.melphalan | 50 | 59 |

*compared with the control (tumor cell cultures)

The reduction in protein synthesis in the cells in the investigated groups was determined in comparison with the control group of cells. As is evident from table 25, MOC.Tegafur in particular inhibits protein biosynthesis.

Exposure of the tumor cell cultures with B-16 melanoma to the MOC substances leads to a change in the morphological structure, a reduction in proliferative activity and inhibition of protein synthesis. This is confirmed by the results of investigations previously obtained in mice through exposure of the B-16 tumor to the complexes.

### Example 15

Pharmacokinetics of the Storage of MOC Products in the Tissues of the Organism

Tumor chemotherapy requires a selective effect of anticancer products. The storage ability of the products in tumor tissue can be calculated for the selectivity. Specifically labeled ³H-Cu(acac)₂ with tegafur were synthesized for the determination. The labeled product was carefully purified from concomitant substances by means of a chromatograph. The total radioactivity of the ³H-Cu(acac)₂Ft is 0.16 microunits.

The distribution and storage of the characterized complex in the organs and tissues was investigated on mice with transplanted AKATON (small bowel cancer). The product was administered in a dosage of 1 200 000 impulses per minute on day 13 after tumor transplantation. Three groups each of 5 mice were formed. The animals were sacrificed 30, 60 and 180 minutes after administration of the product, and the organs were investigated by means of the radioactive impulses using a β counter (βscintigraphy). The results are shown in table 26.

TABLE 26
Progress of storage of ³H—Cu(acac)₂.tegafur in organs
and tissues in the investigated mice (number of
impulses per minute)

| Organs | 30 min | 60 min | 180 min |
|---|---|---|---|
| Brain | 600 | 1200 | 500 |
| Heart | traces | traces | — |
| Lung | 2300 | 2800 | 3000 |
| Liver | 1700 | 3550 | 4000 |
| Kidney | traces | traces | 1200 |
| Small bowel | traces | traces | 3200 |
| Spleen | 1400 | 1200 | 1900 |
| Blood | 3300 | 4700 | 1200 |
| Tumor | 1200 | 1800 | 12 000 |

The maximum storage of the product is observed in the tumor, and this proves targeted transport in the organism.

1-10. (canceled)

11. An antitumor composition comprising at least one complex of the general formula
D₂—M—T
where
D is a β-diketone,
M is a metal atom selected from the group consisting of Cr, Cu, Mn, Fe, Ni, Co, Zn and Mo,
T is a substance having at least one N-, O- or S-containing group, and where M participates in an electron donor-acceptor interaction with T, and M in the complex has a free coordination site.

12. The antitumor composition as claimed in claim 11, wherein D is selected from acetylacetone and dibenzoylmethane.

13. The antitumor composition as claimed in claim 11, wherein T is a substance having at least one NH₂, NH, N, O or S group.

14. The antitumor composition as claimed in claim 11, wherein T is a substance selected from the group consisting of 2,4-dihydroxy-5-fluoropyrimidine, 5-fluoro-1-(tetrahydro-2-furyl) uracil, 2-[bis(2-chloroethyl)-amino]-tetrahydro-2H-1,3,2-oxazephosphorine 2-oxide, 1,2-imido-propanoic amide, 2-hydroxymethyl-5-hydroxy-γ-pyrone, 2,4,6-trimethylpyridine, 2,4,6-tri-2-pyridyl-1,3,5-triazine, 4-[bis(2-chloroethyl)amino]-L-phenylalanine, 2-(3-pyridyl) piperidine, 2-2′-bipyridine, 2-methyl-(5-trimethylbutyl-1-il-ol-3) pyridine, 2-methyl-(3-dimethyl-amino-1-propynyl)pyridine and 2-methyl-5-ethylenepyridine.

15. An antitumor composition of the general formula
D₂—M—T
in which D is acetylacetone, M is Cu and T is a substance selected from the group consisting of
2,4-dihydroxy-5-fluoropyrimidine, 5-fluoro-1-(tetrahydro-2-furyl) uracil, 2-[bis(2-chloroethyl)-amino]-tetrahydro-2H-1,3,2-oxazephosphorine 2-oxide, 1,2-imido-propanoic amide, 2-hydroxymethyl-5-hydroxy-γ-pyrone, 2,4,6-trimethylpyridine, 2,4,6-tri-2-pyridyl-1,3,5-triazine, 4-[bis(2-chloroethyl)amino]-L-phenylalanine, 2-(3-pyridyl) piperidine, 2-2′-bipyridine, 2-methyl-(5-trimethylbutyl-1-il-ol-3) pyridine, 2-methyl-(3-dimethyl-amino-1-propynyl)pyridine and 2-methyl-5-ethylenepyridine.

16. An antitumor composition of the general formula
D₂—M—T
in which D is acetylacetone, M is Mn and T is a substance selected from the group consisting of
2,4,6-trimethylpyridine,
2,4,6-tri-2-pyridyl-1,3,5-triazine, 2-2′-bipyridine, 2-(3-pyridyl) piperidine, 1,2-imidopropanoic amide and 4-[bis(2-chloroethyl)amino]-L-phenylalanine
